(19) United States
(12) Patent Application Publication
(10) Pub. No.: US 2005/0176987 A1
(43) Pub. Date: Aug. 11, 2005

(54) Method for producing vinyl, aryl and heteroaryl acetic acids and derivatives thereof

(76) Inventor: Lukas Goossen, Mulheim an der Ruhr (DE)

Correspondence Address: Norris McLaughlin & Marcus, 30th Floor, 220 East 42nd Street, New York, NY, 10017, US

(21) Appl. No.: 10/471,150

(22) PCT Filed: Mar. 6, 2002
(86) PCT No.: PCT/EP02/02423

(30) Foreign Application Priority Data
Mar. 9, 2001 (DE) 101 11 262.9

(51) Int. Cl. C07C 63/64; C07C 67/02

(52) U.S. Cl. 560/234

(57) ABSTRACT

A process for the preparation of vinyl, aryl and heteroaryl acetic acids and their derivatives by the reaction of vinyl, aryl or heteroaryl boronic acids and their derivatives with α-halo- or α-pseudohaloacetic acids and their derivatives which bear a substituent selected from hydrogen, alkyl or vinyl, aryl and heteroaryl in 2-position in the presence of a palladium catalyst, a base and water. This process enables the preparation of a wide variety of functionalized vinyl, aryl and heteroaryl acetic acids and their derivatives.

The invention relates to a process for the preparation of vinyl, aryl and heteroaryl acetic acids and their derivatives by the reaction of vinyl, aryl and heteroaryl boronic acid derivatives with α-halo- or α-pseudohaloacetic acids or their derivatives which bear a substituent selected from hydrogen, alkyl, vinyl, aryl and heteroaryl in 2-position in the presence of a palladium catalyst, a base and water. This process enables the preparation of a wide variety of functionalized vinyl, aryl and heteroaryl acetic acids and their derivatives.

Methylenecarboxy groups are important functional groups in a number of pharmacologically important compounds, such as the anti-inflammatory agents indomethacin or aclofenac (see, for example, T. Y. Shen, Angew. Chem. 1972, 84, 512-526). Therefore, a mild and efficient method for introducing methylenecarboxy groups into sensitive functionalized molecules would be highly interesting.

Conventional syntheses are multistep in nature, tedious or incompatible with functional groups. In reference books, such as J. March, Advanced Organic Chemistry, 4th Edition, Wiley, New York, 1992, 1281-1282, for the synthesis of arylacetic acids, there is mentioned, in particular, the hydrolysis of benzyl nitrites, which must in turn by synthesized, for example, from benzyl halides. In addition to the multistep nature of the process, a disadvantage of this method is the use of strong acids or bases which results in the cleavage of sensitive functions, such as ester groups.

The Willgerodt reaction of acetophenones, which has also been described, is often unsuitable, for example, because of the intolerance towards further keto substituents. Another disadvantage is the limited availability of acetophenones.

Further known is the reaction of bromobenzenes with chloroacetic acid derivatives in the presence of stoichiometric amounts of silver or copper at 180 to 200° C. A disadvantage of this method is the high temperature, which precludes application with temperature-sensitive compounds, the low yield and the use of stoichiometric amounts of metals which are difficult to recover.

Further known are Friedel-Crafts alkylations of benzenes with α-haloacetic acids and their derivatives. A disadvantage thereof is the fact that, as with all Friedel-Crafts reactions, mixtures of isomers are usually obtained (see, for example, Bull. Soc. Chim. Fr. 1950, 1075-1078).

The reaction of aryl Grignard compounds with α-haloacetic acid derivatives also results in phenylacetic acid derivatives (U.S. Pat. No. 2,250,401). However, a disadvantage thereof is the extremely limited functional group tolerance due to the use of difficult to handle and highly reactive Grignard compounds.

The carbonylation of benzyl halides in the presence of alcohols also yields phenylacetic acid esters. The limited availability of benzyl halides and the necessity of using toxic CO gas are disadvantages of this method.

As alternatives to the mentioned processes, cross-couplings of aryl halides with Reformatsky reagents, tin, copper and other enolates or ketene acetals have recently been described (see, for example, J. Am. Chem. Soc. 1959, 81, 1627-1630; J. Organomet. Chem. 1979, 177, 273-281; Synth. Comm. 1987, 17, 1389-1402; Bull Chem. Soc. Jpn. 1985, 58, 3383-3384; J. Org. Chem. 1993, 58, 7606-7607; J. Chem. Soc. Perkin 1 1993, 2433-2440; J. Am. Chem. Soc. 1975, 97, 2507-2517; J. Am. Chem. Soc. 1977, 99, 4833-4835; J. Am. Chem. Soc. 1999, 727, 1473-78; J. Org. Chem. 1991, 56, 261-263, Heterocycles 1993, 36, 2509-2512, Tetrahedron Lett. 1998, 39, 8807-8810).

However, these methods have limited applicability. Thus, Reformatsky reagents and ketene acetals are tedious to prepare and handle. The use of tin compounds is disadvantageous for toxicological reasons, and the use of stoichiometric amounts of copper causes considerable costs of disposal. The use of enolates is usually possible only if no other enolizable groups are present in the molecule. Therefore, for example, ketones are excluded as substrates for such methods. Some electro-chemical processes are also known (Synthesis 1990, 369-381; J. Org. Chem. 1996, 61, 1748-1755); however, these methods are disadvantageous due to the complicated reaction control and the low yields per space and time.

Further, aryl boronic acid derivatives are known to be advantageous starting materials for cross-couplings because, due to their low toxicity and their insensitiveness towards air and moisture, they are readily stored and easily handled even in a pure form, in contrast to the above mentioned Grignard compounds or aryl zinc compounds. Boronic acid pinacol esters are readily distilled and chromatographed.

Numerous vinyl, aryl or heteroaryl boronic acid derivatives are readily available, for example, by the substitution of aromatics with boric acid esters in the presence of Lewis acids, by the reaction of other vinyl, aryl or heteroaryl metal compounds with boric acid esters, or by palladium-catalyzed coupling reactions, for example, of bispinacoldiboron or pinacolborane with vinyl, aryl or heteroaryl halides or triflates. In the latter reactions, a wide variety of functional groups are tolerated.

Only one example of the reaction of such boronic acid derivatives with α-arylcarbonyl compounds is known, namely the reaction of benzeneboronic acid dibutyl ester with α-bromoacetic acid ethyl ester in the presence of an excess of highly toxic thallium carbonate using tetrakis(triphenylphosphino)palladium as a catalyst at temperatures of clearly higher than 20° C. as described by Suzuki et al. (Chem. Lett. 1989, 1405-1408). With the experimental conditions mentioned, i.e., the use of triphenylphosphine as a ligand and the absolute exclusion of moisture, the use of this base is indispensable. With other, less toxic, bases, no conversions worth mentioning were achieved in control experiments. However, this process is of little interest for commercial applications due to the high price and toxicity of thallium carbonate.

Therefore, there is a need for a process for reacting vinyl, aryl and heteroaryl boronic acid derivatives with α-halo- or α-pseudohaloacetic acids and their derivatives which is characterized by being easily performed, by mild reaction conditions and by the use of inexpensive reagents which are safe to health.

Surprisingly, a process for the preparation of vinyl, aryl and heteroaryl acetic acids and their derivatives from vinyl, aryl and heteroaryl boronic acid derivatives and α-halo- or α-pseudohaloacetic acids or their derivatives has been found which is characterized in that the reaction is performed in the presence of water, an inorganic base and a palladium phosphine complex.

The finding that a low water content in the reaction mixture is not disadvantageous, as with other organometallic reactions, but considerably favors the conversions and selectivities of the reaction, could hardly be foreseen and makes the finding of this process particularly surprising.

By using other phosphine ligands than the triphenylphosphine used by Suzuki, satisfactory conversions and selectivities can be achieved even without the addition of thallium carbonate. Under optimized conditions according to the invention, yields of only 30% were achieved with triphenylphosphine. Sterically more demanding ligands with medium electron densities are ideal for high selectivities at lower temperatures. Especially with tri-1-naphthylphosphine, excellent selectivities of more than 90% are achieved.

In the process according to the invention, vinyl, aryl and heteroaryl acetic acids and their derivatives are obtained in high yields and selectivities already at room temperature. In addition, only toxicologically safe bases are employed. Moreover, hardly any by-products having similar boiling points are formed, but mainly non-toxic inorganic salts are obtained, which is particularly advantageous for the technical operation of the process, since the separation of organic by-products can be problematic and cost-intensive.

The process claimed herein is clearly distinguished by the mentioned processes in which enolates are reacted with aryl halides, since vinyl, aryl or heteroaryl metal compounds are here reacted with α-halo- or α-pseudohaloacetic acids and their derivatives. A particularly advantageous feature is the fact that the readily available boronic acids which are particularly inert towards functional groups can be employed as the metal species.

In the process according to the invention, acetic acids and their derivatives of the series of esters and amides of general formula 1 or 2 which bear a substituent X selected from fluoro, chloro, bromo or iodo or pseudohalo groups selected from arylsulfonyloxy, alkylsulfonyloxy, trifluoromethylsulfonyloxy, alkylcarbonyloxy, arylcarbonyloxy, azido, nitro, diazo, alkyloxycarbonyloxy or aryloxycarbonyloxy in the α-position with respect to the carbonyl group are employed.

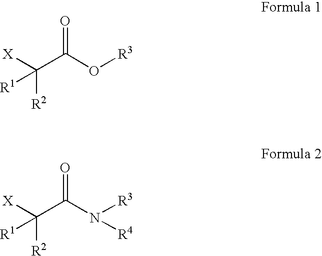

The substituents R¹, R², R³ and, for formula 2, R⁴ are independently selected from hydrogen, linear and branched C₁-C₈ alkyl, vinyl, aryl or heteroaryl selected from pyridine, pyrimidine, pyrrole, thiophene, furan and may themselves bear further substituents selected from linear and branched C₁-C₈ alkyl or C₁-C₈ aryl, linear and branched C₁-C₈ alkyloxy or C₁-C₈ aryloxy, halogenated linear and branched C₁-C₈ alkyl or halogenated C₁-C₈ aryl, linear and branched C₁-C₈-alkyl- or C₁-C₈-aryl-oxycarbonyl, linear and branched C₁-C₈ alkylamino, linear and branched C₁-C₈ dialkylamino, C₁-C₈ arylamino, C₁-C₈ diarylamino, formyl, hydroxy, carboxy, cyano and halo, such as F, Cl, Br and I.

Preferably employed are α-halo- or α-pseudohaloacetic acids and their derivatives of formula 1 or 2 wherein X is bromo or chloro and the substituents R¹, R², R³ and R⁴ may be as described above. More preferably employed are α-halo- or α-pseudohaloacetic acids and their derivatives of formula 1 or 2 wherein X is bromo or chloro which have no hydrogen atoms in the β-position with respect to the carbonyl group. Even more preferably employed are α-bromoacetic acid esters and amides of general formula 1 or 2 in which the substituents R¹ and R² are hydrogens. As the reaction partners, boronic acids and their derivatives of general formula 3 are employed, wherein Z¹ and Z² represent substituents selected from hydroxy, dialkylamino, C₁-C₈ alkyloxy, aryloxy, fluoro, bromo, chloro, iodo. The residues Z₁ and Z₂ may also be interconnected by a C—C bond or through a linear or branched alkyl or aryl bridge. The substituent Ar represents a vinyl, aryl or heteroaryl residue selected from pyridine, pyrimidine, pyrrole, pyrazole, imidazole, oxazole, thiophene, furan, which may itself bear further substituents selected from linear and branched C₁-C₈ alkyl or C₁-C₈ aryl, vinyl or heteroaryl selected from pyridine, pyrimidine, pyrrole, pyrazole, imidazole, oxazole, thiophene, furan, linear and branched C₁-C₈ alkyloxy or C₁-C₈ aryloxy, halogenated linear and branched C₁-C₈ alkyl or halogenated C₁-C₈ aryl, linear and branched C₁-C₈-alkyl- or C₁-C₈-aryl-oxycarbonyl, linear and branched C₁-C₈-alkyl- or C₁-C₈-aryl-carbonyl, linear and branched C₁-C₈ alkylamino, linear and branched C₁-C₈ dialkylamino, C₁-C₈ arylamino, C₁-C₈ diarylamino, formyl, hydroxy, carboxy, cyano, amino and halo, such as F, Cl, Br and I.

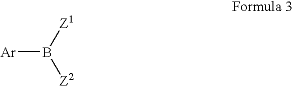

Optionally, the boronic acids may be prepared in situ by the reaction of corresponding vinyl halides, aryl halides or heteroaryl halides or vinyl, aryl or heteroaryl pseudohalides with either a diboron compounds or a borane in the presence of a palladium catalyst according to the prior art.

As bases in the process according to the invention, inorganic bases selected from alkali or alkaline earth hydroxides, carbonates, hydrogencarbonates, oxides, phosphates, hydrogenphosphates, fluorides or hydrogenfluorides are employed, preferably using alkali and alkaline earth phosphates, carbonates or fluorides, more preferably using potassium fluoride, potassium carbonate and potassium phosphate.

In the process according to the invention, from 1 to 10 equivalents of the respective base are employed. Preferably, from 1 to 5 equivalents of the base are employed.

In the process according to the invention, the catalysts are prepared in situ from common palladium(II) salts, such as palladium chloride, bromide, iodide, acetate, acetylacetonate, which may optionally be stabilized by further ligands, such as alkylnitriles, or from Pd(0) species, such as palladium on active charcoal, or tris(dibenzylideneacetone)dipalladium and phosphine ligands PR¹R²R³, wherein R¹ represent substituents selected from hydrogen, linear and branched C₁-C₈ alkyl, vinyl, aryl or heteroaryl selected from pyridine, pyrimidine, pyrrole, thiophene, furan, which may themselves be substituted with further substituents selected from linear and branched C₁-C₈ alkyl or C₁-C₈ aryl, linear and branched C₁-C₈ alkyloxy or C₁-C₈ aryloxy, halogenated linear and branched C₁-C₈ alkyl or halogenated C₁-C₈ aryl, linear and branched C₁-C₈-alkyl- or C₁-C₈-aryl-oxycarbonyl, linear and branched C₁-C₈ alkylamino, linear and branched C₁-C₈ dialkylamino, C₁-C₈ arylamino, C₁-C₈ diarylamino, formyl, hydroxy, carboxy, cyano and halo, such as F, Cl, Br and I. Alternatively, defined palladium complexes may be employed which were previously prepared from the above mentioned ligands in one or more process steps.

In the process according to the invention, from 1 to 20 equivalents of phosphine are employed, based on the amount of palladium employed, from 1 to 4 equivalents being preferably employed.

In the process according to the invention, an amount of catalyst of from 0.001 mole percent to 20 mole percent, based on the acetic acid derivative, is employed. Preferably, an amount of catalyst of from 0.01 mole percent to 3 mole percent is employed.

The process according to the invention is performed at temperatures of from −20° C. to 150° C., preferably from 0° C. to 80° C., and more preferably from 10° C. to 50° C.

The process according to the invention may be performed in the presence of a solvent or in bulk. Preferably, it is performed in the presence of a solvent. Preferred solvents are water, saturated aliphatic hydrocarbons, alicyclic hydrocarbons, aromatic hydrocarbons, alcohols, amides, sulfoxides, sulfonates, nitriles, esters or ethers.

As the solvent, there may be employed, for example, pentane, hexane, heptane, octane, cyclohexane, benzene, toluene, xylenes, ethylbenzene, mesitylene, dioxane, tetrahydrofuran, diethyl ether, dibutyl ether, methyl t-butyl ether, diisopropyl ether, diethylene glycol dimethyl ether, methanol, ethanol, propanol, isopropanol, methyl acetate, ethyl acetate, t-butyl acetate, dimethylformamide, diethylformamide, N-methylpyrrolidone, dimethylacetamide, dimethyl sulfoxide, sulfolane, acetonitrile, propionitrile or water.

More preferably employed are aromatic hydrocarbons, amides, esters and ethers. Even more preferably employed are ethers.

The process according to the invention is performed in the presence of water. Preferably, it is performed in the presence of from 0.1 to 100 equivalents of water, based on the acetic acid derivative. In this amount, the water contained in the solvent and in the reagents is to be taken into account. It is particularly preferred to add from 1 to 50 equivalents of water. Even more preferably, from 2 to 20 equivalents of water is added.

The process according to the invention is preferably performed by starting with the catalyst, the α-halo- or α-pseudohaloacetic acid derivative, the base and part of the solvent and metering in the boronic acid derivative in a further portion of the solvent.

For isolating the vinyl, aryl and heteroaryl acetic acids and their derivatives prepared according to the invention, the reaction mixture is processed upon completion of the reaction, preferably by distillation and/or extraction. Preferably, the reaction mixture is processed by extraction and subsequent distillation.

### EXAMPLES

### Example 1

Synthesis of bromobutylphenylacetic acid: Palladium acetate (67.3 mg, 0.30 mmol), tri-1-naphthylphosphine (371 mg, 0.90 mmol), 4-bromobutyl bromoacetate (2.74 g, 10.0 mmol) and potassium phosphate (10.61 g, 50.0 mmol) were charged into a flask. The reaction vessel was then flushed with argon and sealed with a septum cap. A solution of benzeneboronic acid (1.46 g, 12.0 mmol) in THF (40 ml) and water (0.36 ml, 20 mmol) was added, and the reaction was stirred at room temperature for some hours. The progress of the reaction was monitored by means of thin-layer chromatography. After the reaction was complete, the reaction mixture was poured into water (300 ml) and extracted three times with 100 ml each of dichloromethane. The combined organic fractions were washed with water, dried over magnesium sulfate and filtered. The residue was distilled in a high vacuum. As a main fraction, a colorless oil (2.41 g; 89%) having a boiling point of 91° C./0.01 mbar was obtained and identified as the expected reaction product. ¹H NMR (300 MHz, CDCl₃, 25° C., TMS): δ=7.33-7.26 (m, 5H); 4.12 (t, ³J (H,H)=6 Hz, 2H); 3.62 (s, 2H); 3.38 (t, 33 (H,H)=6 Hz, 2H); 1.87 (m, 2H); 1.79 (m, 2H) ppm; ¹³C NMR (75 MHz, CDCl₃, 25° C., TMS): δ=171.5; 134.0; 129.2; 128.6; 127.1; 63.8; 41.4; 32.9; 29.2; 27.2 ppm; MS (70 eV): m/z (%): 270(6) [M⁺], 191(4), 179(4), 136(23), 91(100); HRMS: calc. for C₁₂H₁₅BrO₂ [M⁺]: 270.02555; found: 270.02546; anal. calc. for C₁₂H₁₅BrO₂ (271.16): C, 53.16; H, 5.58; N, 0.0; found: C, 52.96; H, 5.65; N, 0.0.

### Examples 2-16

In Examples 2 to 16, 1 mmol each of bromoacetic acid ethyl ester was reacted with 1.2 mmol of benzeneboronic acid in the presence of 5 mmol of the specified base, 0.03 mmol of palladium acetate (in Example 15: tris(dibenzylideneacetone)-dipalladium(0)), 0.09 mmol of the corresponding phosphine ligand and 2 mmol of water. Four milliliters each of the specified solvents was employed. The products were purified by column chromatography and characterized by means of NMR and GC-MS. The results are summarized in Table 1.

TABLE 1
Influence of the reaction parameters on the conversion and selectivity

| | | | | Conversion | Selectivity |
|---|---|---|---|---|---|
| Example | Ligand | Base | Solvent | (%) | (%) |
| 2 | PPh₃ | K₂CO₃ | THF | 95 | 34 |
| 3 | P(m-tol)₃ | K₂CO₃ | THF | 75 | 3 |
| 4 | P(o-tol)₃ | K₂CO₃ | THF | 100 | 86 |
| 5 | P(o-EtPh)₃ | K₂CO₃ | THF | 100 | 51 |
| 6 | P(m-xyl)₃ | K₂CO₃ | THF | 100 | 80 |
| 7 | P(mes)₃ | K₂CO₃ | THF | 80 | 80 |
| 8 | P(t-Bu)₂biph | K₂CO₃ | THF | 100 | 67 |
| 9 | P(nap)₃ | K₂CO₃ | THF | 100 | 88 |
| 10 | BINAP | K₂CO₃ | THF | <5 | — |
| 11 | P(o-tol)₃ | KF | THF | 100 | 78 |
| 12 | P(o-tol)₃ | Et₃N | THF | 100 | 36 |
| 13 | P(o-tol)₃ | K₂CO₃ | DMF | 90 | 14 |
| 14 | P(o-tol)₃ | K₂CO₃ | CH₃CN | 82 | 35 |
| 15^{a} | P(o-tol)₃ | K₂CO₃ | THF | 100 | 89 |
| 16 | P(nap)₃ | K₃PO₄ | THF | 100 | 91 |

^{a}(dba)₃Pd₂ instead of palladium acetate

### Examples 17-30

Examples 17 to 30, 1 mmol each of the respective bromoacetic acid derivative Br—CH₂COX was reacted with 1.2 mmol of the respective boronic acid Ar—B(OH)₂ in the presence of 5 mmol of potassium phosphate, 0.03 mmol of palladium acetate, 0.09 mmol of tri-1-naphthylphosphine and 2 mmol of water in 4 ml of THF at 20° C. to form the products Ar—CH₂COX. The products were purified by column chromatography and characterized by means of NMR and GC-MS. The results are summarized in Table 2.

TABLE 2
Examples 17 to 30

| Example | Ar | X | Yield (%)^{a} |
|---|---|---|---|
| 17 | phenyl | OEt | 85 |
| 18 | o-tolyl | OEt | 90 |
| 19 | 1-naphthyl | OEt | 80 |
| 20 | p-MeO-phenyl | OEt | 84 |
| 21 | p-acetylphenyl | OEt | 79^{c} |
| 22 | p-tolyl | OEt | 90 |
| 23 | m-chlorophenyl | OEt | 70^{b} |
| 24 | p-formylphenyl | OEt | 67^{c} |
| 25 | m-nitrophenyl | OEt | 40^{b} |
| 26 | m-AcNH-phenyl | OEt | 63 |
| 27 | 2-thienyl | OEt | 33^{b} |
| 28 | 2-fluorophenyl | OEt | 31^{c} |
| 29 | phenyl | N(C₅H₁₀) | 81 |
| 30 | phenyl | O(C₄H₈)Br | 72 |

^{a}isolated yields;
^{b}KF instead of K₃PO₄;
^{c}K₂CO₃ instead of K₃PO₄

### Examples 31-36

In Examples 31 to 36, 1 mmol each of the respective bromoacetic acid derivative Br—CH₂COX was reacted with 1.2 mmol of the respective boronic acid Ar—B(O₂C₆H₁₂) in the presence of 5 mmol of potassium phosphate, 0.03 mmol of palladium acetate, 0.09 mmol of tri-1-naphthylphosphine and 2 mmol of water in 4 ml of THF at 20° C. The products were purified by column chromatography and characterized by means of NMR and GC-MS. The results are summarized in Table 3.

TABLE 3
Examples 31 to 36

| Example | Ar | X | Yield (%)^{a} |
|---|---|---|---|
| 31 | phenyl | OEt | 87 |
| 32 | o-tolyl | OEt | 75 |
| 33 | 1-naphthyl | OEt | 68 |
| 34 | p-MeO-phenyl | OEt | 76 |
| 35 | p-acetylphenyl | OEt | 60 |
| 36 | phenyl | O(C₄H₈)Br | 68 |

^{a}isolated yields;
^{b}K₂CO₃ instead of K₃PO₄

1. A process for the preparation of vinyl, aryl or heteroaryl acetic acids or derivatives thereof comprising reacting vinyl, aryl or heteroaryl boronic acids or derivatives thereof with α-halo- or α-pseudohaloacetic acids or derivatives thereof which bear a substituent selected from hydrogen, aryl or alkyl in 2-position, wherein the reacting is performed in the presence of an inorganic base, water and a palladium complex.

2. The process according to claim 1, wherein said reaction is performed with 2-halo- or α-pseudohalo-acetic acids or derivatives thereof according to formula 1 or 2: 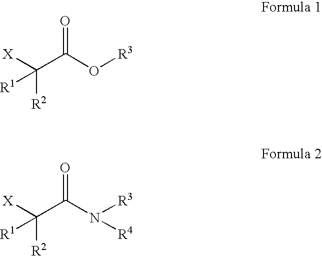
wherein R¹, R², R³ and R⁴ are independently substituents selected from hydrogen, linear and branched C₁-C₈ alkyl, vinyl, aryl or heteroaryl selected from pyridine, pyrimidine, pyrrole, thiophene, furan, which may themselves bear further substituents selected from linear and branched C₁-C₈ alkyl or C₁-C₈ aryl, linear and branched C₁-C₈ alkyloxy or C₁-C₈ aryloxy, halogenated linear and branched C₁-C₈ alkyl or halogenated C₁-C₈ aryl, linear and branched C₁-C₈-alkyl- or C₁-C₈-aryl-oxycarbonyl, linear and branched C₁-C₈ alkylamino, linear and branched C₁-C₈ dialkylamino, C₁-C₈ arylamino, C₁-C₈ diarylamino, formyl, hydroxyl, carboxy, cyano, F, Cl, Br and I.

3. The process according to claim 2, wherein α-chloro- or α-bromoacetic acids or their derivatives of formula 1 or 2 which do not have hydrogen atom in an α-position with respect to the halogen atom are employed.

4. The process according to any of claims 2 to 3 claim 3, wherein said reacting is performed with a-bromoacetic acid esters or esters or amides of general formula 1 or 2 in which the substituents R¹ and R² are hydrogens.

5. The process according to claim 1, wherein said reacting is performed with boronic acids or boronic acid derivatives of general formula 3 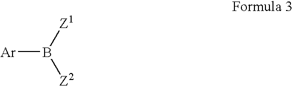
wherein
Z¹ and Z² represent substituents selected from hydroxy, dialkylamino, C₁-C₈ alkyloxy, aryloxy, fluoro, bromo, chloro, iodo, which may be interconnected by a C—C bond or through a linear or branched alkyl chain, a vinyl or aryl group;
Ar represents aryl, vinyl or heteroaryl residue selected from pyridine, pyrimidine, pyrrole, thiophene, furan, which may itself bear further substituents selected from linear and branched C₁-C₈ alkyl or C₁-C₈ aryl, linear and branched C₁-C₈ alkyloxy or C₁-C₈ aryloxy, halogenated linear and branched C₁-C₈ alkyl or halogenated C₁-C₈ aryl, linear and branched C₁-C₈ alkyl- or C₁-C₈-aryl-oxycarbonyl, linear and branched C₁-C₈ alkylamino, linear and branched C₁-C₈ dialkylamino, C₁-C₈ arylamino, C₁-C₈ diarylamino, formyl, hydroxy, carboxy, cyano, F, Cl, Br and I.

6. The process according to claim 1, wherein said palladium complex is produced from a palladium (II) salt or a palladium (0) compound and a phosphine ligand PR¹R²R³, wherein R¹, R² and R³ represent substituents selected from hydrogen, linear and branched C₁-C₈ alkyl, aryl, vinyl or heteroaryl selected from pyridine, pyrimidine, pyrrole, thiophene, furan, which may themselves be substituted with further substituents selected from linear and branched C₁-C₈ alkyl or C₁-C₈ aryl, linear and branched C₁-C₈ alkyloxy or C₁-C₈ aryloxy, halogenated linear and branched C₁-C₈ alkyl or halogenated C₁-C₈ aryl, linear and branched C₁-C₈-alkyl- or C₁-C₈-aryl-oxycarbonyl, linear and branched C₁-C₈ alkylamino, linear and branched C₁-C₈ dialkylamino, C₁-C₈ arylamino, C₁-C₈ diarylamino, formyl, hydroxy, carboxy, cyano, F, Cl, Br and I.

7. The process according to claim 1, wherein a palladium complex is employed which is generated from a palladium (II) salt or a palladium (0) compound and a triarylphosphine ligand in which at least one of the aryl rings in ortho position is substituted by a substituent selected from linear and branched C₁-C₈ alkyl or C₁-C₈ aryl, linear and branched C₁-C₈ alkyloxy or C₁-C₈ aryloxy, linear and branched halogenated C₁-C₈ alkyl or halogenated C₁-C₈ aryl, linear and branched C₁-C₈-alkyl- or C₁-C₈-aryl-oxycarbonyl linear and branched C₁-C₈ alkylamino, linear and branched C₁-C₈ dialkylamino, C₁-C₈ arylamino, C₁-C₈ diarylamino, formyl, hydroxyl, carboxy, cyano, F, Cl, Br and I, wherein said substituent in ortho position may additionally be part of an anellated aryl or heteroaryl ring selected from pyridine, pyrimidine, pyrrole, thiophene, furan.

8. The process according to claim 7, wherein tri-1-naphthylphosphine is used as said phosphine ligand.

9. The process according to any of claims 6 to 8 claim 6, wherein from 1 to 20 equivalents of phosphine is used, based on the amount of palladium.

10. The process according to any of claims 1 to 9 claim 1, wherein from 0.001 to 20 mole percent of the palladium complex is used, based on the acetic acid derivative.

11. The process according to claim 6, wherein the preparation of the palladium complex is effected in situ.

12. The process according to claim 1, wherein an alkali or alkaline earth fluoride, hydrogenphosphate, hydrogencarbonate, phosphate or fluoride is employed as said inorganic base.

13. The process according to claim 12, wherein potassium carbonate, phosphate or fluoride is employed as said inorganic base.

14. The process according to claim 1, wherein said reacting is performed in the presence of one to twenty equivalents or water, based on the acetic acid derivative.

15. The process according to claim 1, wherein said reacting is performed in an ether as the solvent.

16. The process according to claim 1, wherein said reacting is performed at a temperature of between 0° C. and 80° C.
